(19) United States
(12) Patent Application Publication
(10) Pub. No.: US 2017/0165442 A1
(43) Pub. Date: Jun. 15, 2017

(54) PERFORATED FACE MASK FOVE FENESTRATED CPAP ORAL VENTILATION ELIMINATOR

(71) Applicant: Bernard Robert Borbely, St. Augustine, FL (US)

(72) Inventor: Bernard Robert Borbely, St. Augustine, FL (US)

(21) Appl. No.: 14/965,719

(22) Filed: Dec. 10, 2015

(51) Int. Cl. A61M 16/06

(52) U.S. Cl. CPC A61M 16/0683

(57) ABSTRACT

The face support mask for respiratory conditions of the first preferred embodiments consists of a piece of flexible material with at least two perforations positioned so that they will be over the mouth of a user, where the piece of flexible material is attached to a releasable coupler on both lateral sides, where the releasable couplers are designed to releasably attach the piece of flexible material to lateral face straps. The face support mask for respiratory conditions of the preferred embodiments is preferably designed to apply restraining pressure to a user's lips and cheeks to prevent air from escaping from a user's mouth while they are sleeping, while also allowing the user to sigh, make noise, sneeze, cough, move their mouth or carry out other intentional actions while they are wearing the face support mask and are awake.

### BRIEF DESCRIPTION OF THE FIGURES

FIG. 1 is a schematic representation of the system of the preferred embodiments by itself, with at least two perforations over the mouth area.

FIG. 2 is a schematic representation of the system of the preferred embodiments, where the face support mask is in use and releasably attached to the lateral face straps of another device, where at least the hook portion of a hook and loop fastener is used as the releasable coupler, and at least four perforations are included.

FIG. 3 is a schematic representation of the system of the preferred embodiments, where the face support mask is in use and releasably attached to lateral face straps that are integrally connected to the bottom of the piece of flexible material, where at least the hook portion of a hook and loop fastener is used as the releasable coupler, and at least four perforations are included.

FIG. 4 is a schematic representation of the system of the preferred embodiments viewed from the side, where the face support mask is in use and releasably attached to the lateral face straps of another device, where the second strap is coupled to the lateral face straps and wrapped around the back of the user's head.

FIG. 5 is a schematic representation of the system of the preferred embodiments viewed from the side, where the face support mask is in use and releasably attached to lateral face straps that are integrally connected to the bottom of the piece of flexible material, where the second strap is coupled to the lateral face straps and wrapped around the back of the user's head.

FIG. 6 is a schematic representation of the system of the preferred embodiments, where the all portions of the lips are restrained and the piece of flexible material extends in at least 1 cm in all directions around the user's closed lips, where the face support max is releasably coupled to the lateral face straps of another device.

FIG. 7 is a schematic representation of the system of the preferred embodiments, where the all portions of the lips are restrained and the piece of flexible material extends in at least 1 cm in all directions around the user's closed lips, where the face support max is releasably coupled to the lateral face straps integrally attached to the bottom of the piece of flexible material.

FIG. 8 is a schematic representation of the system of the preferred embodiments viewed from the back, where the two sides of the second strap are coupled to one another behind the user's head.

FIG. 9 is a schematic representation of the system of the preferred embodiments, showing the preferred variation where the flexible piece of material is integrally attached on its bottom side to lateral face straps, shown laid out fully.

FIG. 10 is a schematic representation of the system of the preferred embodiments, showing the preferred variation having porous material to create the at least one perforation and the subsequent airflow.

### DESCRIPTION OF THE PREFERRED EMBODIMENTS

The following description of the preferred embodiments of the invention is intended to enable someone skilled in the prior art to make and use this invention, but is not intended to limit the invention to these preferred embodiments.

### 1. First Preferred Embodiment

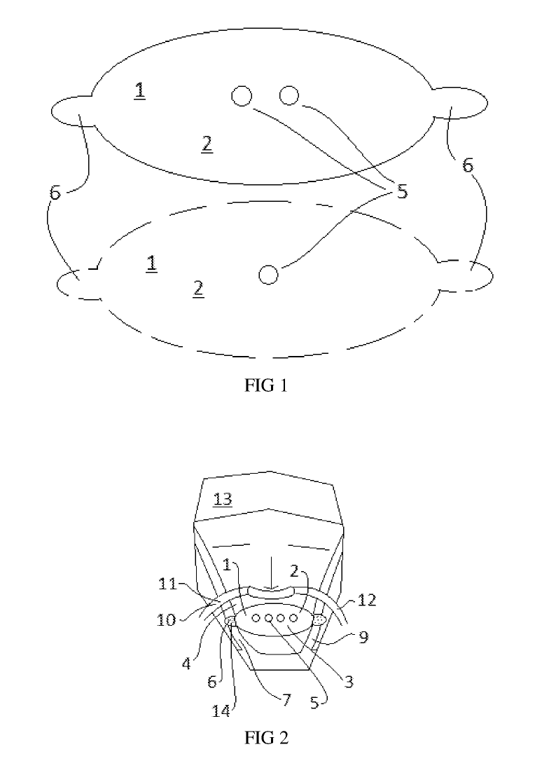

As shown in FIG. 1, the face support mask for respiratory conditions 1 of the first preferred embodiments consists of a piece of flexible material 2 with at least two perforations positioned so that they will be over the mouth 3 of a user, where the piece of flexible material 2 is attached to a releasable coupler on both lateral sides, where the releasable couplers 6 are designed to releasably attach the piece of flexible material 2 to lateral face straps. The face support mask for respiratory conditions 1 of the preferred embodiments is preferably designed to apply restraining pressure to a user's lips 8 and cheeks 4 to prevent air from escaping from a user's mouth 3 while they are sleeping, while also allowing the user 13 to sigh, make noise, sneeze, cough, move their mouth 3 or carry out other intentional actions while they are wearing the face support mask 1 and are awake.

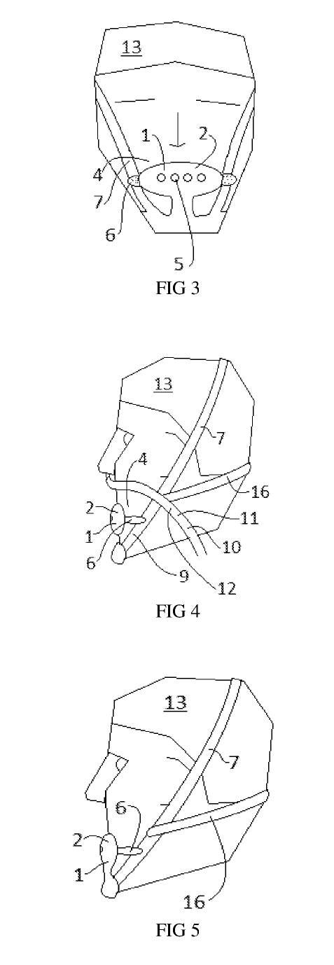

As shown in FIG. 1, the at least two perforations in the piece of flexible material 2 are preferably positioned so that when the face support mask 1 is worn by a user, the perforations will be located over at least one of the user's mouth 3 and the user's lips 8. The perforations preferably allow the user 13 to carry out intentional actions while they are awake, such as sighing, coughing, sneezing, making noise, moving their mouth 3, or other actions. The perforations may, however, be used for any suitable reasons. In a preferred variation, there is at least one perforation located over at least one of the user's mouth 3 and the user's lips 8. In another preferred variation, a porous material 17 is used in the mask over at least one of the user's mouth 3 and the user's lips 8, where the at least one perforation is created by the pores in the material. As shown in FIG. 1, the piece of flexible material 2 is attached on both lateral sides to a releasable coupler. As shown in FIGS. 2 and 3, the releasable couplers 6 preferably releasably attach the piece of flexible material 2 to lateral face straps. As shown in FIG. 2, in one preferred variation the lateral face straps 7 may be part of another device. As shown in FIG. 3, in another preferred variation the lateral face straps 7 may be integrally attached to the bottom of the piece of flexible material 2, creating a stand-alone system. In one preferred variation, the lateral face straps 7 are attached to a positive pressure ventilation mask 9. In another preferred variation, the lateral face straps 7 are attached to a CPAP face mask 11. In another preferred variation, the lateral face straps 7 are attached to a bilevel positive pressure vetilation mask 12. The lateral face straps 7 may, however, be part of any system and may be present for any suitable reason.

As shown in FIGS. 2 and 3, in one preferred variation the releasable couplers 6 are at least the hook portion of hook and loop fasteners 14. The hook portion of hook and loop fasteners 14 may be capable of directly attaching to the material used in the lateral face straps 7 by hooking into the material. In one preferred variation an industrial grade hook fastener is used. In another preferred variation, the releasable coupler may be a hook and loop fastener attached to a flexible strap, such that the flexible strap can wrap around the lateral face straps 7 and the hook portion of the fastener can releasably attach to the loop portion of the fastener on the opposite end of the of the flexible strap. In another preferred variation, the releasable coupler can be at least one of: a snap fastener, a buckle fastener, and a tie fastener. The releasable couplers 6 may, however, be any suitable type of releasable coupler.

As shown in FIGS. 2 and 3, in one preferred variation there are at least four perforations in the flexible piece of material. In one preferred variation, the combined area of these at least four openings is at least .15 square inches. In another preferred variation, the combined area of these at least four openings is at least .3 square inches. The combined area of the openings may, however, be any suitable amount. There may, however, be any suitable number of perforations.

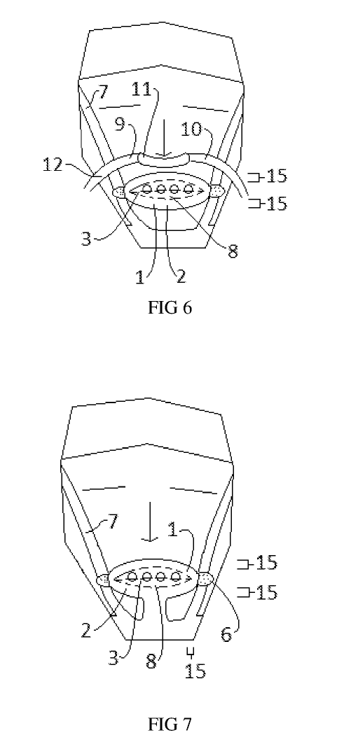

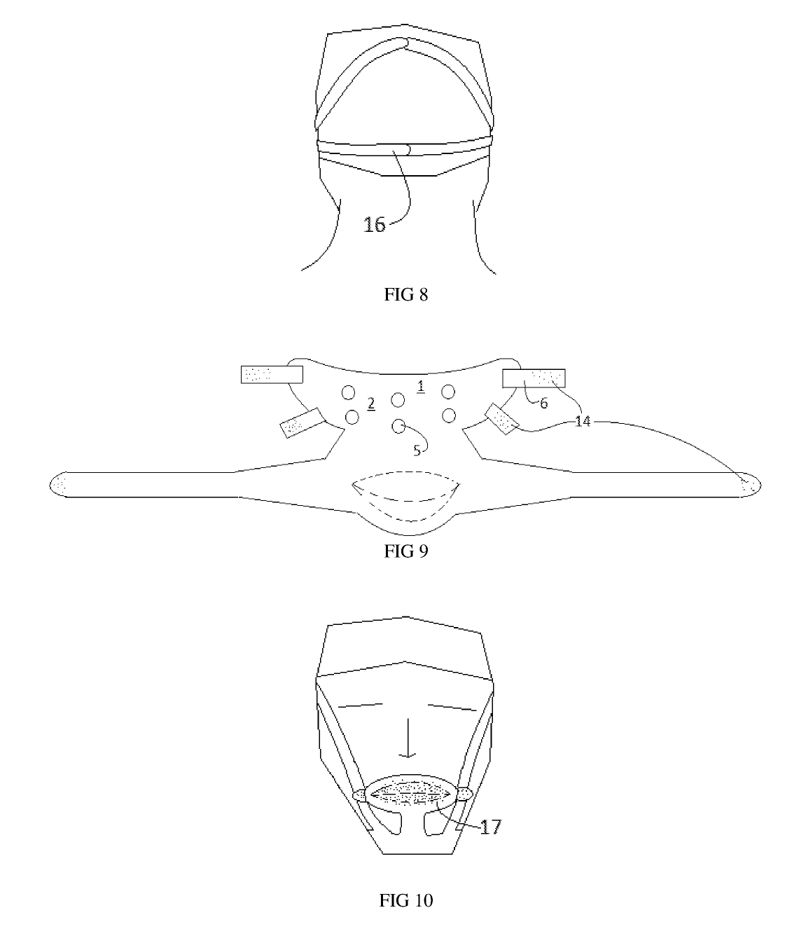

As shown in FIGS. 6 and 7, the face support mask 1 preferably restrains all portions of a user's lips 8. In one preferred variation, the face support mask 1 is used with a positive pressure ventilation device 10. In such a device, the pressure applied through the mask of the device may be applied through tubing to an interface with the user's nose, however this positive pressure gas may leak through a user's mouth 3 and out of a user's lips 8. This causes the intended positive pressure to dissipate, counteracting the effects of the positive pressure ventilation device 10. Preferably the face support mask 1 applies restraining pressure to all portions of a user's lips 8 when the piece of flexible material 2 is releasably attached to the lateral face straps. This restraining pressure prevents the lips 8 from moving outward as a result of the pressure from the positive pressure ventilation device 10, and thus prevents gas from leaking out of the user's lips 8. Preferably the face support mask 1 also restrains a portion of the user's cheeks 4, preventing the user's cheeks 4 from billowing out and further ensuring that the user's lips 8 will not allow gas to leak through them. The positive pressure ventilation device 10 may be at least one of a CPAP device 11, a bilevel positive pressure ventilation device 12, and any other positive pressure ventilation device 10. Preferably the flexible piece of material extends at least 1 cm 15 past the user's lips 8 in all directions when worn by the user 13 and when the user's lips 8 are closed. This preferably allows the face support mask 1 to provide restraint to all portions of the user's lips 8 as well as portions of the user's cheeks 4. Prior art devices do not allow the user 13 to comfortably carry out intentional actions including sighing, speaking, coughing, sneezing, and moving their mouth 3, while at the same time providing sufficient support to the user's lips 8 and cheeks 4 to prevent gas from leaking out of the user's lips 8. Prior art devices also do not attach to the straps of existing devices. In another preferred variation, the piece of flexible material 2 is integrally attached to lateral face straps, and the lateral face straps 7 releasably attach to each other above and behind the user's ears. In this preferred variation, the face support mask 1 is capable of being used by itself, without a positive pressure ventilation device 10. In this preferred variation the face support mask 1 may be used to reduce or prevent snoring in a user. As shown in FIG. 9, in this preferred variation the system may further comprise a chin strap integrally attached to the lateral face straps. The face support mask 1 may, however, provide any suitable restraint to at least one of the lips 8 and the cheeks 4 of the user. The face support mask 1 may, however, be used in conjunction with any suitable device. The face support mask 1 may, however, be used for any suitable reason.

As shown in FIGS. 4, 5, and 8, in one preferred variation a second strap 16 16 is releasably attached to the lateral face straps 7 and wraps behind the user's head, where the two sides of the second strap 16 releasably attached to each other. In this variation, the second strap 16 preferably provides tension in opposition to the tension that may be created by releasably attaching the face support mask 1 to the lateral straps. This preferably increases the amount of tension that can be applied to the face support mask 1, allowing the face support mask 1 to provide greater restraining pressure to at least one of the user's lips 8 and cheeks 4. The second strap 16 is preferably adjustable in length, by a means that may include a friction buckle or any other suitable means of adjusting the length of a strap. The second strap 16 may be releasably coupled to the lateral face straps 7 with at least the hook portion of a hook and loop fastener 14. The two sides of the second strap 16 may releasably couple to each other behind the user's head using at least one of: a hook and loop fastener, a tie fastener, a snap fastener, and a buckle fastener. The second strap 16 may, however, have any suitable means for releasably attaching to the lateral face straps. The second strap 16 may, however, have any suitable means for releasably coupling the two sides of the second strap 16. The second strap 16 may, however, be used for any suitable reason. The system of the first preferred embodiments may, however, not use any form of second strap 16.

The piece of flexible material 2 is preferably a neoprene material. In an alternative variation, the piece of flexible material 2 is at least one of: a cotton fabric, a wool fabric, a nylon fabric, a synthetic fabric, a flexible piece of polymer, and a bamboo fabric. In the preferred variation where the piece of flexible material 2 is integrally attached on its lower edge to the lateral face straps, the lateral face straps 7 preferably are made of a material that the hook side of a hook and loop fastener 14 can attach to. The lateral face straps 7 may, however, be made of any suitable material. The piece of flexible material 2 may, however, be made of any suitable material. The flexible piece of material is preferably capable of being trimmed to accommodate different users. The flexible piece of material may, however, have any suitable means for accommodating different users or none at all.

As a person skilled in the art will recognize from the previous detailed description and from the figures and claims, modifications and changes can be made to the preferred embodiments of the invention without departing from the scope of this invention defined in the following claims.

I Claim:

1) A face support mask for respiratory conditions comprising a section of flexible material designed to fit over the mouth and at least part of the cheeks of a user, wherein the section of flexible material has at least one perforation over the area adapted to cover the mouth of the user, wherein a releasable coupler is attached to both lateral sides of the pieces of material, wherein the releasable couplers are adapted to releasably couple the piece of flexible material to lateral face straps.

2) The face support mask for respiratory conditions of claim 1, wherein the lateral face straps are integrally attached to the face support mask at the bottom of the face support mask, wherein the lateral face straps wrap around the user's head and releasably couple to each other above and behind the user's ears.

3) The face support mask for respiratory conditions of claim 1, wherein the lateral face straps are a attached to a separate device.

4) The face support mask for respiratory conditions of claim 3, wherein the lateral face straps are attached to a positive pressure ventilation mask.

5) The face support mask for respiratory conditions of claim 3, wherein the lateral face straps are attached to a CPAP device mask.

6) The face support mask for respiratory conditions of claim 3, wherein the lateral face straps are attached to a bilevel positive pressure ventilation device mask.

7) The face support mask for respiratory conditions of claim 4, wherein the releasable couplers are at least the hook component of hook and loop fasteners.

8) The face support mask for respiratory conditions of claim 5, wherein the releasable couplers are at least the hook component of hook and loop fasteners.

9) The face support mask for respiratory conditions of claim 1, further comprising at least four perforations, wherein the combined area of the openings of the at least four perforations is at least .15 square inches.

10) The face support mask for respiratory conditions of claim 4, further comprising at least four perforations, wherein the combined area of the openings of the at least four perforations is at least .15 square inches.

11) The face support mask for respiratory conditions of claim 7, further comprising at least four perforations, wherein the combined area of the openings of the at least four perforations is at least .3 square inches.

12) The face support mask for respiratory conditions of claim 10, wherein the face support mask restrains all portions of a user's lips.

13) The face support mask for respiratory conditions of claim 12, wherein the section of flexible material covers an area extending at least 1 cm beyond a user's lips in all directions when the user's lips are closed.

14) The face support mask for respiratory conditions of claim 2, wherein the face support mask restrains all portions of a user's lips, wherein the section of flexible material covers an area extending at least 1 cm beyond a user's lips in all directions when the user's lips are closed.

15) The face support mask for respiratory conditions of claim 14, further comprising at least four perforations, wherein the combined area of the openings of the at least four perforations is at least .3 square inches.

16) The face support mask for respiratory conditions of claim 7, wherein the face support mask restrains all portions of a user's lips.

17) The face support mask for respiratory conditions of claim 12, wherein the section of flexible material covers an area extending at least 1 cm beyond a user's lips in all directions when the user's lips are closed, further comprising at least four perforations, wherein the combined area of the openings of the at least four perforations is at least 0.15 square inches.

18) The face support mask for respiratory conditions of claim 14, further comprising a second strap adapted to releasably couple to one lateral face strap and wrap around the back of the user's head to couple to the lateral face strap on the opposite side of the user's face.

19) The face support mask for respiratory conditions of claim 15, further comprising a second strap adapted to releasably couple to one lateral face strap and wrap around the back of the user's head to couple to the lateral face strap on the opposite side of the user's face.

20) The face support mask for respiratory conditions of claim 1, wherein the at least one perforation is provided by a porous material covering the mouth of the user, wherein the at least one perforation is created in the form of the pores in the material.
